(19) United States
(12) Patent Application Publication
(10) Pub. No.: US 2008/0177330 A1
(43) Pub. Date: Jul. 24, 2008

(54) Self-locking screws for medical implants

(76) Inventors: James D. Ralph, Bethlehem, PA (US); Thomas N. Troxell, Pottstown, PA (US); Mark Michels, Glen Mills, PA (US)

Correspondence Address: NORRIS MCLAUGHLIN & MARCUS, P.A., P O BOX 1018, SOMERVILLE, NJ, 08876, US

(21) Appl. No.: 11/585,718

(22) Filed: Oct. 24, 2006

(51) Int. Cl. A61B 17/56

(52) U.S. Cl. 606/290

(57) ABSTRACT

Self-locking surgical screw assemblies are described for fastening a medical implant to a bone. The invention also includes combinations of self-locking screws and implants, devices implanted with self-locking screws and related methods which insure the stable attachment of the implant to the bone. Each self-locking surgical screw assembly is comprised of a screw having a non-threaded head affixed to a shank, a threaded portion on the shank, a neck on the shank and a locking device externally affixed to or disposed on the head and/or neck. The locking device lockingly engages the head and/or neck of the screw when the screw is in fastening engagement with the medical implant and the bone.

### BACKGROUND OF THE INVENTION

1. Field of the Invention

The present invention has to do with surgical fasteners which are implanted in the body. In particular, the invention has to do with self-locking surgical screws which do not back out after they are implanted. The screws are particularly suitable for affixing plates and other implant devices in a patient.

2. The Related Art

Screws presently used for fastening implants to a bone rely on the screw obtaining solid purchase in the bone and holding down the implant tightly against the bone. The compression of the implant to the bone provides a rigid connection which allows the forces (stresses) to be transferred from the bone to the implant. If this compression is lost, then the transference of force to the implant is reduced or even lost completely. But bones can be weak or have weak portions and motion or repeated forces can cause the screws to back out, thus loosening the implant, potentially making it ineffective and/or causing pain or infection, or impinging on internal structures in the patient. An additional surgical procedure may be required as a result.

Bone generally has an outer cortical shell which is hard and strong. The portion of the bone under the cortical shell is cancellous bone, which is a much softer material. This characteristic of bone structure creates problems in surgery because much of the length of the screw often is fixed in this weaker cancellous bone. Conventional surgical screws can loosen and become unstable, for example, they may rotate over time in the loosening direction (termed “backout”) or pull out requiring a second or revision surgery or the patient may have weak bone tissue or bones weakened by disease such as osteoporosis so that the bones are not strong enough to hold a tightly seated screw.

Self-locking surgical screw systems have been designed to overcome these problems because a self-locking screw is prevented from rotating and thereby backing out of a bone. U.S. Pat. No. 5,951,558, for example, describes a bone fixation device comprising a fixation plate and screws in combination with a screw locking or blocking mechanism which is arranged to prevent the screws from backing out after they are passed through the plate and screwed into the bone. This device requires a combination of screws with a specially designed plate. The design prevents gross screw backout but still allows a small amount of loosening which can be clinically significant.

Other devices for locking surgical screws in place require a threaded screw hole in the implant device which receives a specially designed screw with a threaded head. In these devices the screw must be inserted at a pre-determined fixed angle with respect to the implant. A few examples of devices embodying this design are illustrated in U.S. Pat. Nos. 5,709,686, 5,954,722, 6,623,486 and 6,821,278.

The self-locking screw assemblies of the present invention do not require a specially designed implant device or threaded screw holes in the implant device. The screws of the invention can be used in place of standard implant screws in conventional implant devices. Another advantage of the screws of the invention is that they can be installed on an angle that is most suitable to the surgical procedure and, if desired, they can be used in combination with screws that are not self-locking. For example, in a bone plate requiring several screws, some of the screws can be conventional and others can be self-locking.

### SUMMARY OF THE INVENTION

The invention has to do with self-locking screws for fastening a medical implant to a bone, combinations of self-locking screws and implants, devices implanted with self-locking screws and related methods. Each self-locking screw assembly is comprised of a non-threaded head affixed to a shank, a threaded portion on the shank, a neck on the shank between the head and threaded portion and a locking device externally affixed to or disposed on the head, the neck or both the head and the neck. The locking device lockingly engages the head and/or neck of the screw when the screw is in fastening engagement with the medical implant and the bone. When the locking device engages only the head, the neck can be the portion of the screw where the head transitions to the threaded portion.

The surgical applications for use of the self-locking screws of the invention are numerous. They are useful in any application where a surgeon might need a means to repair or rebuild a bone, attach cartilage or a tendon to a bone, and the like. For example, the screws can be inserted through the top tibial trays, can be used to hold down trauma plates, artificial joints, other plates and mesh materials, acetabulum cups, and the like.

### BRIEF DESCRIPTION OF THE DRAWINGS

The drawings are intended to be illustrative, are not drawn to scale and are not intended to limit the scope of the claims to the embodiments depicted.

FIG. 1 is an expanded perspective view of a retaining ring type self-locking surgical screw assembly and a bone plate. FIGS. 1A, 1B, 1C, 1D, 1E and 1F illustrate various elements and views of the FIG. 1 assembly including in FIG. 1D a section view of the assembly in use.

FIG. 2 is an expanded perspective view of a compression type self-locking surgical screw assembly. FIG. 2A illustrates an alternate embodiment of the compression collar used with the FIG. 2 embodiment.

FIG. 3 is an expanded perspective view of a plug type self-locking surgical screw assembly. FIGS. 3A, 3B and 3C illustrate various elements and views of the FIG. 3 assembly including in FIG. 3B a section view of the assembly in a bone plate.

FIG. 4 is an expanded perspective view of a spherical split ring type self-locking surgical screw assembly. FIG. 4A is an elevation view of an assembled FIG. 4 embodiment.

FIG. 5 is a perspective view of a flange type self-locking screw assembly having wedge shaped tabs located below the head. FIGS. 5A, 5B, 5C and 5D illustrate various elements and views of the FIG. 5 assembly, including FIG. 5C which illustrates the assembly installed in a bone plate.

FIG. 6 is an expanded elevation view of a dual split ring type self-locking screw assembly. FIG. 6A is a perspective view of an assembled FIG. 6 embodiment, FIG. 6B is a section view of an assembled FIG. 6 embodiment and FIG. 6C illustrates an alternative embodiment of a ring that can be used with the FIG. 6 assembly.

FIG. 7 is an expanded perspective view of a Bellville washer type self-locking screw assembly. FIG. 7A is a perspective view of an assembled FIG. 7 embodiment and FIG. 7B is a section view of an assembled FIG. 7 embodiment.

FIG. 8 is an expanded perspective view of a dual plug type self-locking screw assembly. FIG. 8A is a partially expanded partial section view of the FIG. 8 embodiment and FIG. 8B is a section view of an assembled FIG. 8 embodiment. FIG. 8C illustrates the FIG. 8 embodiment in use.

FIG. 9 is an expanded perspective view of a crushable collar type self-locking screw assembly. FIG. 9A is a perspective view of an assembled FIG. 9 embodiment and FIG. 9B is a section view illustrating the FIG. 9 embodiment in use.

FIG. 10 is a perspective view of a locking collar type self-locking screw assembly. FIG. 10A is a bottom view of the FIG. 10 embodiment and FIG. 10B illustrates the embodiment in section.

### DESCRIPTION OF THE PREFERRED EMBODIMENTS

The purpose of the self-locking screw assemblies of the invention is to prevent the screws from loosening and backing out of the bone and/or the medical implant. In a typical application, the medical implant is a plate for joining broken bones. Backing out would allow the plate to come loose which could lead to non-fusion (non union) or improper fusion (malunion) of the pieces of bone being joined. There is also the problem of screws which have backed out irritating the soft tissue or impinging upon major arteries, veins or other internal body structures. The self-locking screw assemblies of the invention are intended to be used with existing medical implants which are attached to the bone of a patient by screws. Thus, the screws of the invention are intended to work with standard (non-locking) hole details found in the majority of bone plates and other implant devices manufactured today. This style of screw can simply be added to a set of hospital inventory without requiring the addition of matching plates.

The self-locking screws of the invention are designed to permit angulation and subsidence while still preventing back out. In some cases, postoperatively it is advantageous to allow the screws to change angle relative to the bone plate to accommodate remodeling of the bony ends during the fusion process. This remodeling can lead to minute shortening of the bony ends. In cases such as this, allowing the construct to subside and maintain bony contact, rather than causing the construct and the bone to be rigidly held apart, can aid in achieving fusion.

Self-locking screw assemblies of the invention can be made of various biocompatible materials and combinations of biocompatible materials. For example, the head, shaft, threads and locking device of a particular self-locking screw assembly can be made from the same materials or different materials so that one portion will be absorbed by the body more quickly than another or one portion will be absorbed and the other will not. In this manner, a self-locking screw can be constructed that remains tightly locked during the time required for bones to fuse, but then the locking device degrades over time to permit removal or allow the implant to be unloaded to prevent stress shielding of the bone. Stress shielding is the redistribution of load (and consequently the stress on the bone) as a result of an implant. Metallic implants can be significantly stiffer than bone and as a result, the normal stresses are altered in that region, sometimes causing complications. In the case of a bone plate used to bridge a fractured bone, the bone plate redistributes the load around the fracture. While this is helpful in the early stages of bone healing, as bony fusion progresses and stability across the fracture increases, it would be beneficial for the implant to bear progressively less of the load. Alternatively, the screws or portions of the screws and/or locking device can be coated as described in our copending application Ser. No. 11/025,213, filed Dec. 29, 2004, the disclosure of which is incorporated by reference herein in its entirety. Other variations and combinations of materials that can be used to make the products of the invention will be apparent to those having skill in the art. Suitable materials include biocompatible metals, alloys, polymers and reinforced polymers, both resorbable and nonresorbable, as well as organic materials such as collagen or alloplastic substances which are commonly used in surgical implants of all kinds. Such materials include materials that have sufficient strength to meet the objectives of the invention and that have been approved by the United States Food and Drug Administration (FDA) for surgical implant applications.

Generally speaking, there are three main types of alloys used in orthopedic implants today, titanium alloys, cobalt alloys and stainless steel alloys. An exhaustive list is available on the FDA website which also provides the reference numbers and effective dates of the ASTM or ISO standards for the materials. Some examples include unalloyed and alloyed titanium, molybdenum, chromium, cobalt, tungsten, aluminum, niobium, manganese or vanadium in various combinations as alloys or components of alloys, various stainless steels and other iron alloys, aluminum oxides, zirconium oxides, tantalum and calcium phosphates.

Numerous types of polymers also are employed to make implants and many of these are identified not only on the FDA website mentioned above but also on the ASTM website. Examples of suitable polymers include polyetheretherketone (PEEK), epoxys, polyurethanes, polyesters, polyethylenes, vinyl chlorides, polysulfones, polytetrafluoro-ethylene (PTFE), polycarbonates, polyaryletherketone (PAEK), polyoxymethylene, nylon, carbon fiber polyester, polyetherketoneetherketoneketone (PEKEKK), silicones, hydrogels and the like. When a polymer is used, a small wire or other material can be incorporated in the main body of the base for purposes of x-ray detection.

The foregoing lists of materials may have application in some embodiments of the present invention but not in others as will be apparent to those skilled in the art based on requirements of strength, flexibility, machinability and the like for the particular application. The lists are intended to be illustrative and not exhaustive. Other materials and new materials may be employed based upon the principles of the invention as set forth herein.

For purposes of this specification, the term “polymer” is defined as any biocompatible non-bioabsorbable polymer, copolymer, polymer mixture, plastic or polymer alloy having sufficient strength to withstand without failure the torques and stresses that a self-locking screw of the invention would normally be subjected to during surgery or in the body.

Bioabsorbable or resorbable material can also be used to make all or a portion of one or more of the component parts of the self-locking screw assemblies of the invention and/or the bioabsorbable material can be applied as a partial or complete coating on such component parts.

The terms “bioabsorbable material” and “resorbable material” as used herein includes materials which are partially or completely bioabsorbable in the body.

Suitable bioabsorbable and resorbable materials (also referred to herein as “bioabsorbables”) include polyglycolide, poly(lactic acid), copolymers of lactic acid and glycolic acid, poly-L-lactide, poly-L-lactate; crystalline plastics such as those disclosed in U.S. Pat. No. 6,632,503 which is incorporated herein by reference; bioabsorbable polymers, copolymers or polymer alloys that are self-reinforced and contain ceramic particles or reinforcement fibers such as those described in U.S. Pat. No. 6,406,498 which is incorporated herein by reference; bioresorbable polymers and blends thereof such as described in U.S. Pat. No. 6,583,232 which is incorporated herein by reference; copolymers of polyethylene glycol and polybutylene terephthalate; and the like. The foregoing list is not intended to be exhaustive. Other bioabsorbable materials can be used based upon the principles of the invention as set forth herein.

Bioactive materials can be admixed with the bioabsorbable or resorbable materials, impregnated in such materials and/or coated on the outer surface thereof and/or coated on any other portion of the products of the invention. These materials can include, for example, bioactive ceramic particles, bone chips, polymer chips, capsules or reinforcement fibers and they can contain, for example, antimicrobial fatty acids and related coating materials such as those described in Published U.S. Patent Application No. 2004/0153125 A1; antibiotics and antibacterial compositions; immunostimulating agents; tissue or bone growth enhancers and other active ingredients and pharmaceutical materials known in the art.

The products of the invention can be made by molding, heat shrinking or coating a bioabsorbable material on a base or substrate which has been provided with attachment means such as those described in our pending patent application Ser. No. 11/025,213 filed Dec. 29, 2004 which is incorporated herein as referenced above. When the bioabsorbable material will have functional mechanical properties, the bioabsorbable material can be molded onto a suitable metallic or polymeric substrate in the desired shape. Alternatively, the bioabsorbable material also can be coated, shrink wrapped or molded onto a substrate. If necessary, the bioabsorbable material can be machined to the desired shape and/or dimensions.

The self-locking screw assemblies of the invention are inserted through a hole in a medical implant device. They can comprise self-drilling screws which are screwed into the operating area of a patient with little or no pre-drilling or they can comprise self-tapping screws which can be implanted in pre-drilled holes in the operating area of a patient. The operating area is usually in bone but it can be in cartilage and bone when a means (e.g., a washer, plate, bracket, wire or equivalent) is used to hold the cartilage down. If a pre-drilled hole is used, it is sized to accommodate the self-locking screw so that the implant device will ultimately be implanted in the manner deemed most desirable by the surgeon. It will be understood that sizing the hole means the hole itself and any countersinking that may be desired are drilled in a manner that will cause the self-locking screw and implant device to be securely affixed in the patient during surgery.

As will be apparent to those skilled in the art, the sizes of the self-locking screws of the invention can be varied over a broad range to meet their intended applications. In most cases the self-locking screw assemblies of the invention will work with plates presently on the market. Additionally, in most cases the head style of the self-locking screws which we have illustrated as spherical can be changed to match head styles presently on the market as will be apparent to those having ordinary skill in the art. The drive style can be a slotted or hexagon drive, cruciform or others, as will also be apparent to those having skill in the art.

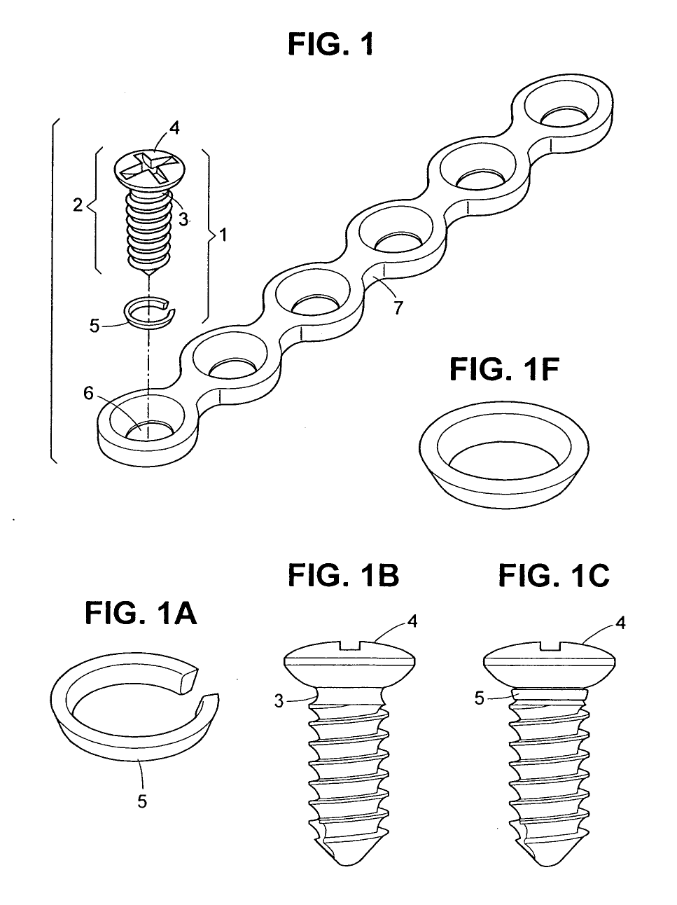

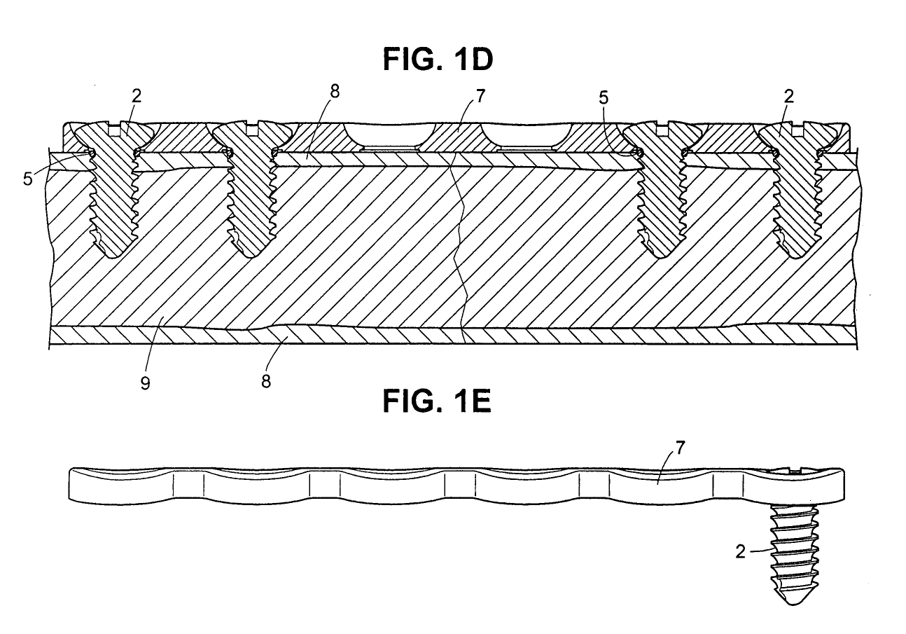

FIG. 1 illustrates an expanded view of a retaining ring self-locking surgical screw assembly 1 which comprises a bone screw 2 with a neck in the form of a groove 3 underneath the head 4 and a retaining (or locking) ring 5 which is wedge shaped and split as illustrated in more detail in FIG. 1A. Either piece may be made from metal, bioabsorbables and/or polymers and/or coated metals, bioabsorbables or polymers as explained above. Another possibility would be to make portions of the screw assembly from an organic material such as collagen. For example, collagen could easily be used to make the retaining ring 5. Collagen, as well as some polymers, is hydrophilic and they swell after absorbing water. This swelling action can augment the mechanical expansion of the locking mechanism. Furthermore, when the material used to make the retaining ring is pliable it can be continuous rather than split as illustrated by ring 5a in FIG. 1F. Ring 5a can be solid or tubular as long as it is sufficiently pliable to be slipped over the threads and be retained in groove 3.

FIG. 1B is an elevation of the bone screw without retaining ring 5 and FIG. 1C is an elevation of the bone screw with the retaining ring 5. The ring is inserted into the groove 3 in the bone screw 2 and the self-locking screw assembly 1 is driven through a screw hole 6 in a bone plate 7 until it is seated in the hole in the plate. As the ring 5 passes through the hole in the plate it compresses and after clearing the plate it expands to lock itself into the plate.

FIG. 1D illustrates in section a plate 7 affixed with screws 2 to a bone comprised of a cortical shell 8 and a cancellous portion 9. The ring 5 is compressed against the inside of the screw hole in the bone plate.

FIG. 1E is an elevation of a plate 7 having a screw 2 in one of the screw holes thereof.

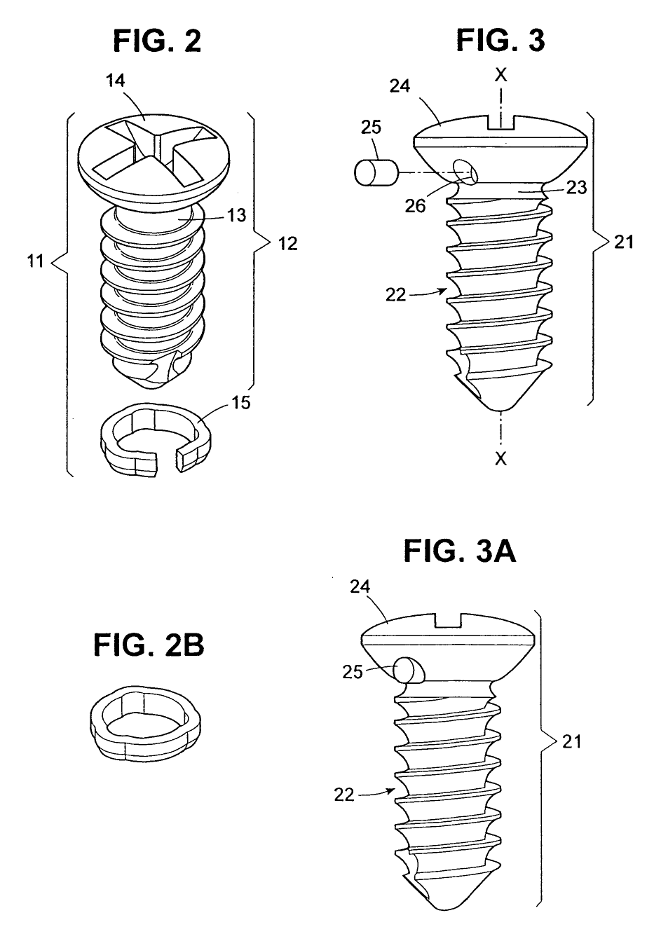

FIG. 2 illustrates an expanded view of a compression type self-locking surgical screw assembly 11 comprising a bone screw 12 with a neck in the groove 13 under the head 14 and a compression collar 15. The bone screw 12 may be made from metal, bioabsorbables and/or polymer and the compression collar 15 may be made from a softer metal, bioabsorbables, collagen and/or polymer. The collar 15 is placed in the groove 13 and the assembly 11 is inserted through a screw hole in a bone plate and screwed into a bone. As with the ring of FIG. 1, the collar can be continuous rather than split if it is sufficiently pliable as illustrated by collar 15a in FIG. 2A. Collar 15a can be solid or tubular. As the compression collar passes through the hole in the plate the collar compresses and wedges into the hole.

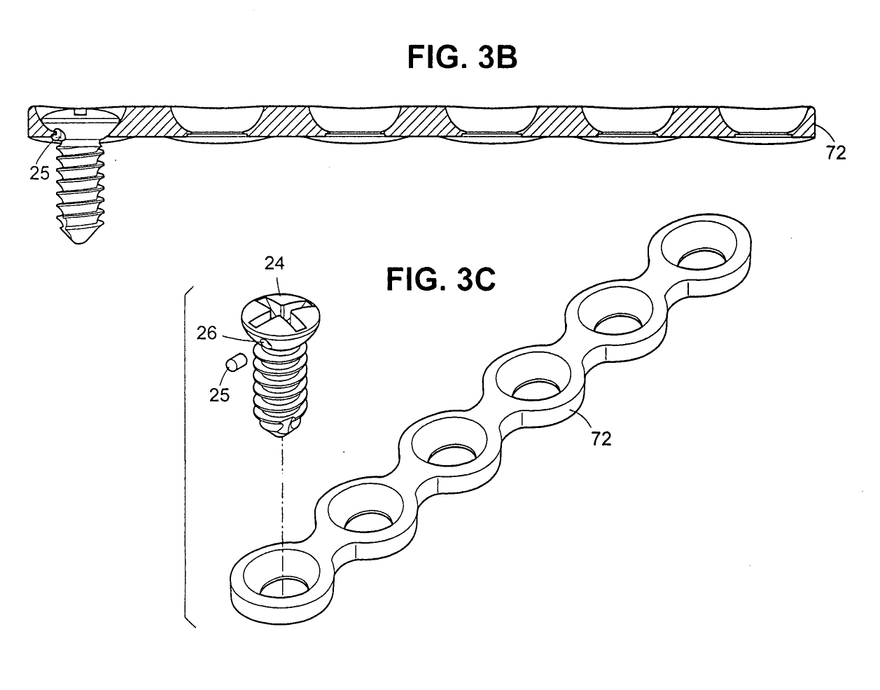

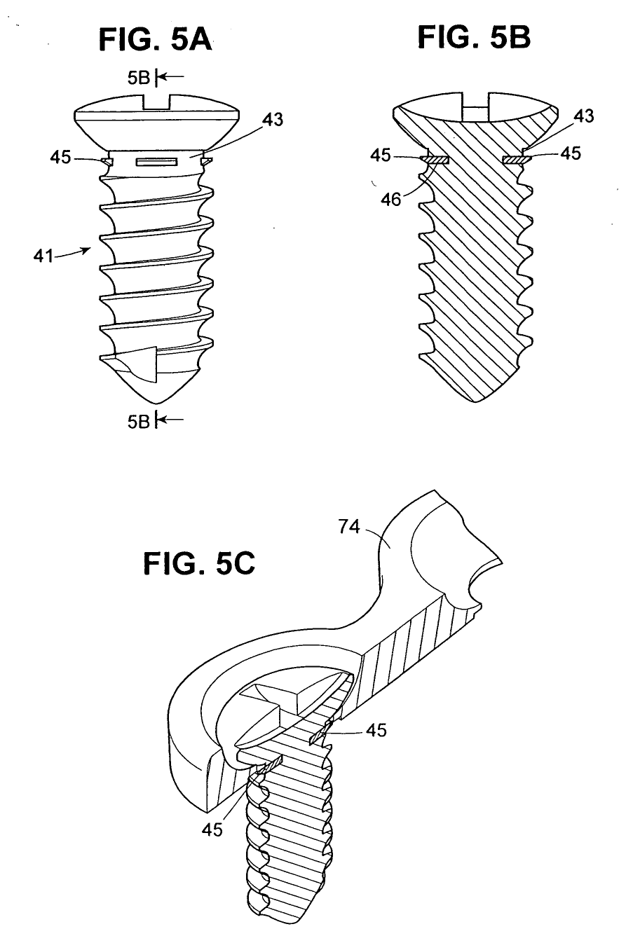

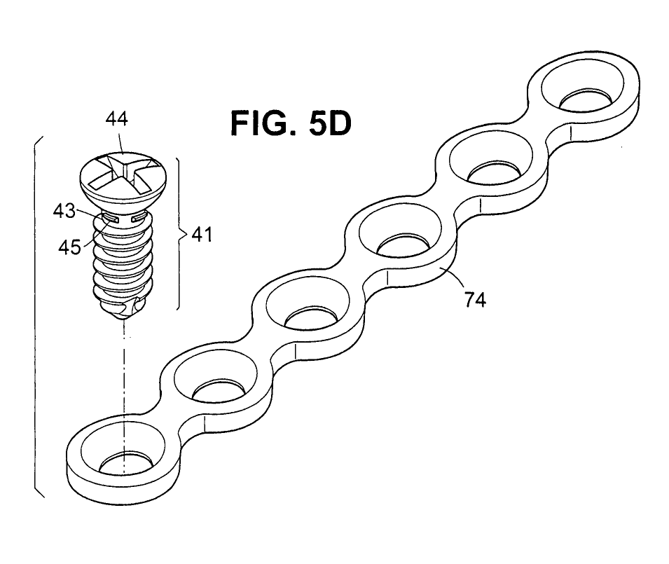

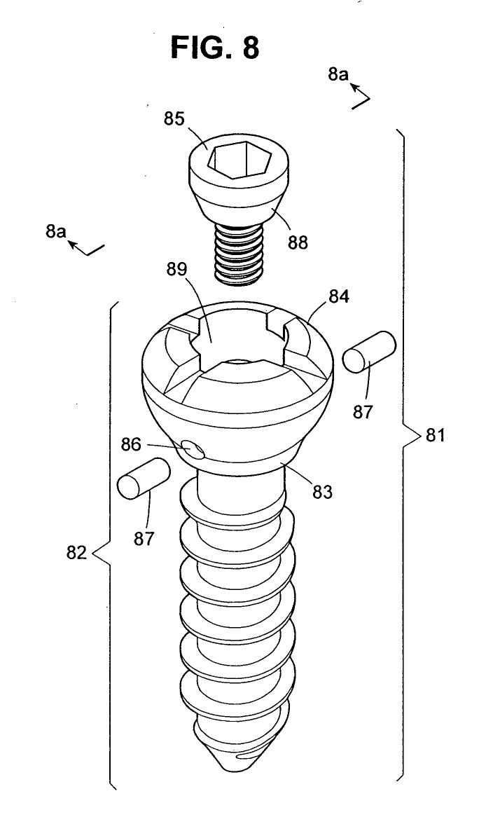

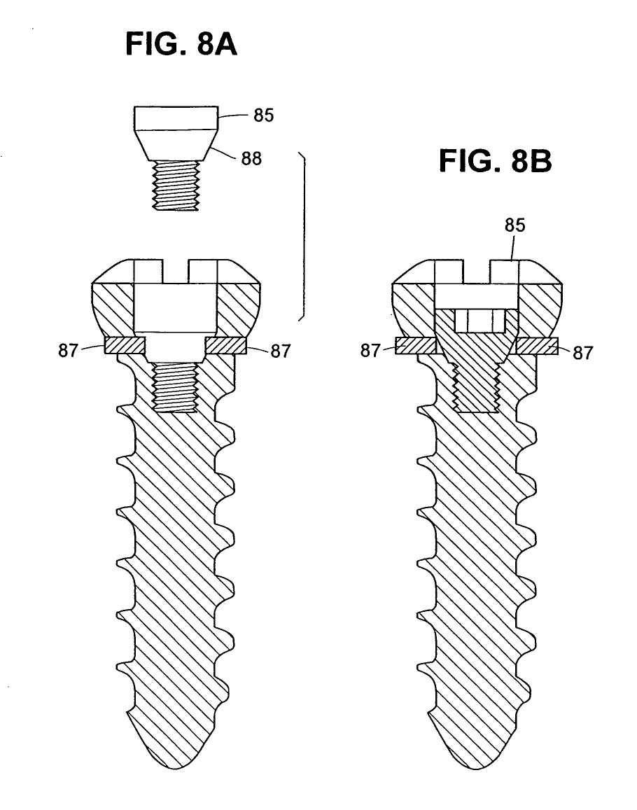

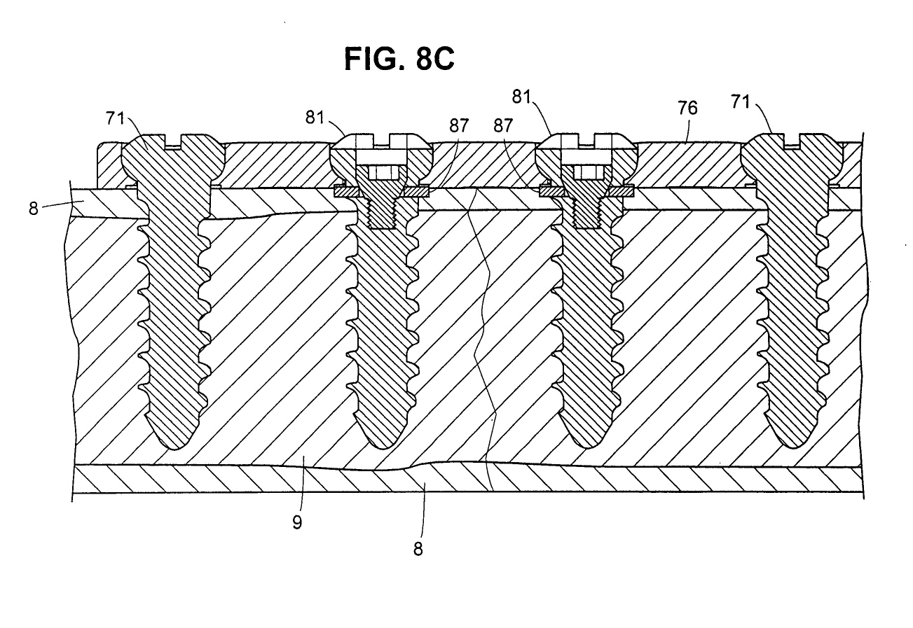

FIG. 3 is an expanded view of a plug type self-locking surgical screw assembly 21 which comprises a bone screw 22 with a spherical head 24 and a small hole 26 in the side of the spherical head and partially in neck 23. The hole 26 is perpendicular to the central axis x-x of the bone screw 22. A plug 25 which is molded, pressed or glued into the hole 26 in the bone screw 22 and protrudes slightly is illustrated in FIG. 3A. Alternatively, there can be two or several holes 26 with plugs 25 therein. (See, for example, FIG. 5 which illustrates four wedges or FIG. 8 which illustrates two plugs.) The hole or holes 26 can be completely in the head or completely in the neck, rather than partially in the neck and head as illustrated, as will be apparent to those having skill in the art. The bone screw may be made from metal, bioabsorbables and/or polymer and the plug is made from a bioabsorbable material, a polymer material or an organic material such as collagen. The screw assembly is passed through a screw hole in a medical implant such as a bone plate and screwed into the bone. As the plug in the screw enters the screw hole in the bone plate, the plug is compressed into the hole and provides an interference fit so that the screw will be locked in place and won't back out as a result of bodily motion. FIG. 3B illustrates a plate 72 with the self-locking surgical screw assembly affixed therein. The plug 25 is partially deformed thereby creating the desired interference fit.

A typical screw hole in a bone plate has a larger opening on the upper side of the plate and a smaller opening on the bottom side (the side adjacent the bone) and the hole is shaped to accommodate the head of the screw. If the plug passes partially through the bone plate it will also interfere with the smaller portion of the hole at the bottom of the plate thereby causing the screw to resist back out. With this design, however, the screw can be backed out with the forces applied by a tool such as a screw driver. An expanded view of this embodiment is illustrated in FIG. 3C.

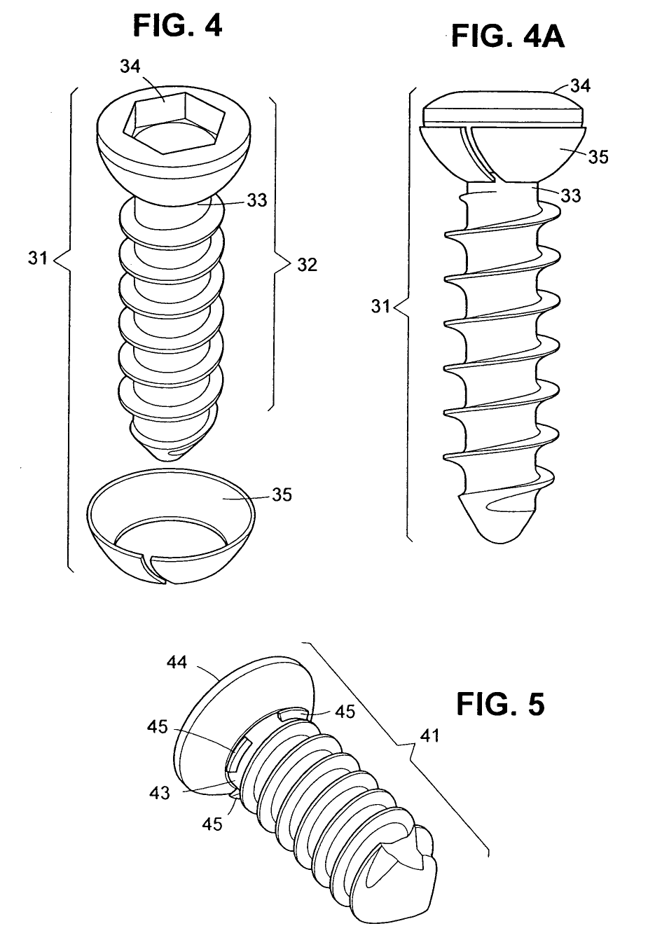

FIG. 4 is an expanded view of a spherical split ring type self-locking surgical screw assembly 31 comprising a bone screw 32 with a neck in the form of a narrow groove 33 below a spherical head 34. A spherical split washer 35 is also illustrated. FIG. 4A illustrates the assembly 31 with washer 35 disposed on the neck and head. The bone screw and washer may be made from metal, bioabsorbables and/or polymer and the washer also can be made from an organic material such as collagen. The washer is preassembled on the screw and the assembly is passed through the hole in the plate and screwed into the bone. As the screw head enters the screw hole in the plate, the washer compresses and provides an interference fit with the plate hole.

FIG. 5 illustrates in perspective a flange type self-locking surgical screw assembly 41 having four wedge shaped tabs 45 located on a neck 43 underneath the spherical head 44. Variants of this embodiment can have one wedge shaped tab or two or more than two such tabs as will be apparent to those having skill in the art. The screw may be made from metal, bioabsorbables and/or polymer and the tabs also may be made from metal, bioabsorbables and/or polymer or an organic material such as collagen. An elevation view of assembly 41 is illustrated in FIG. 5A and a section view is illustrated in FIG. 5B. The section view shows tabs 45 installed in slots 46 in the neck 43. The tabs can be held in place by mechanical pressure, adhesive or the like. As the screw passes through a screw hole in a bone plate and is screwed into the bone, the wedges are compressed or deflected until they pass through the smaller portion of the hole at the bottom of the bone plate at which time they spring back to prevent the screw from backing out of the plate as illustrated in FIG. 5C with a plate 74. If the screw is placed at an angle rather than perpendicular to the plate, at least one of the tabs will pass through the screw hole in the plate and prevent backing out. An expanded perspective view of the assembly 41 and plate 74 is illustrated in FIG. 5D.

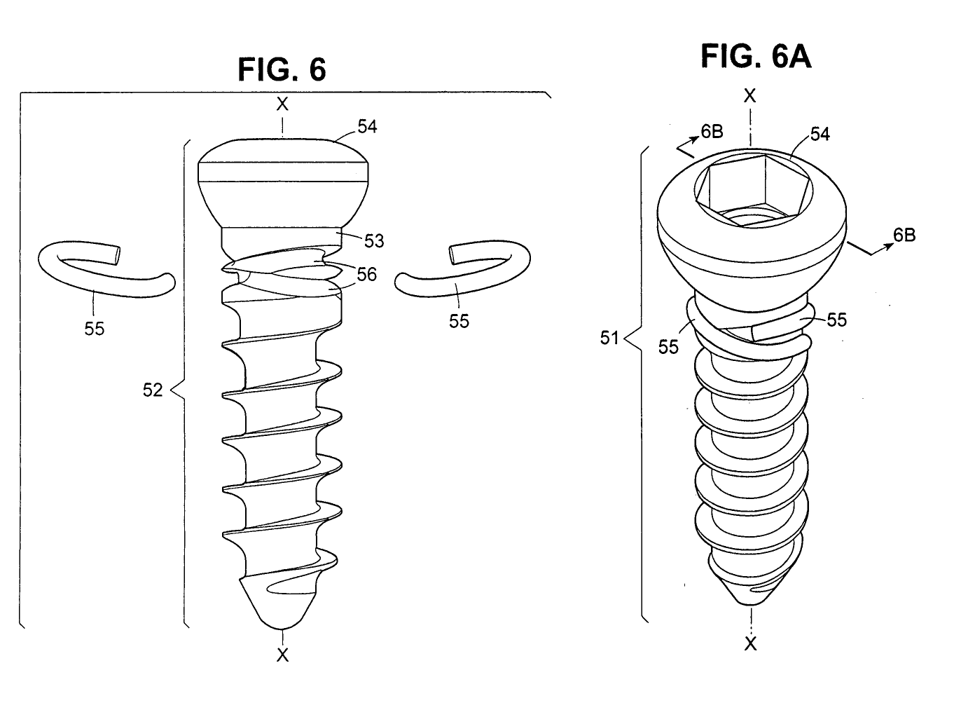

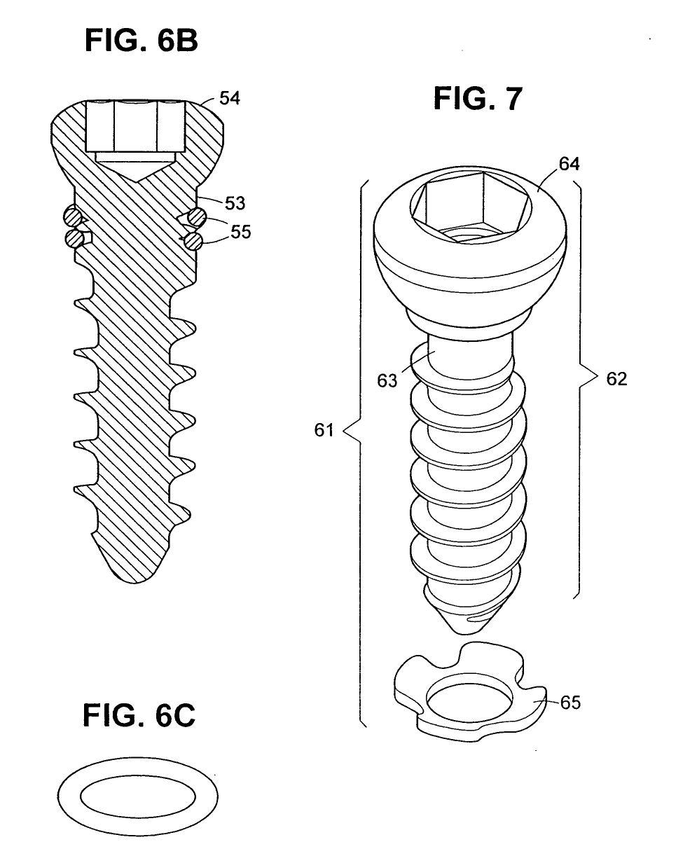

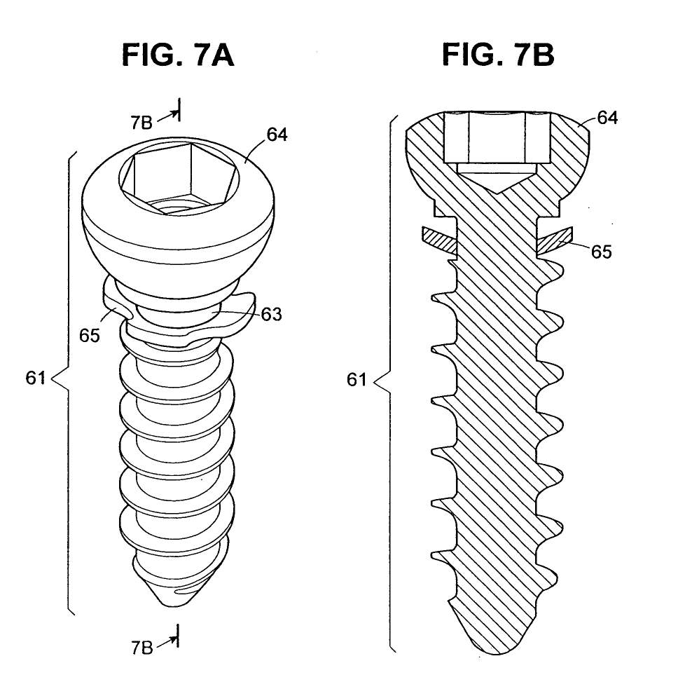

FIG. 6 is an expanded view of a dual split ring type self-locking surgical screw assembly 51 which comprises a spherical head bone screw 52 with two grooves 56 in the neck 53 of the screw, the grooves being at angles of less than 90° to the x-x central axis of the screw. Two round split rings 55 fit into the grooves 56. Both the screw and the rings may be made from metal, bioabsorbables and/or polymer and the rings also may be made from an organic material such as collagen. The rings are assembled into the grooves in bone screw 52 with the open ends of the rings at the two intersections of the rings as illustrated in FIG. 6A (i.e., the rings do not cross over one another). Alternatively, when the material used to make the rings is sufficiently pliable, the rings can be continuous rather than split as illustrated by ring 55a in FIG. 6C. Ring 55a can be solid or tubular and when solid rings are used they do cross over one another. FIG. 6B illustrates the assembly in section. As the screw passes through the screw hole in the bone plate and is screwed into the bone, the rings compress into the grooves and then expand back when they pass through the plate, retaining the screw in the plate.

FIG. 7 is an expanded view of a Bellville washer type self-locking surgical screw assembly 61 which comprises a spherical head bone screw 62 with a groove 63 in the neck directly under the head 64 and a Bellville type washer 65 which is located in the groove and free to move up and down the groove. A perspective view with the washer disposed on the neck of the screw is illustrated in FIG. 7A and a section view is provided by FIG. 7B. Both the screw and the washer may be made from metal, bioabsorbables and/or polymer and the washer also may be made from an organic material such as collagen. The screw is inserted through a screw hole in the plate and screwed into the bone. The washer compresses as it passes through the hole in the plate and expands when it clears the far side, retaining the screw in the plate. The groove allows for variations in the plate thickness.

FIG. 8 is an expanded view of a dual plug type self-locking surgical screw assembly 81 comprising a bone screw 82 with two holes 86 in the sides of the head 84 at the bottom (and can partially extend into the neck 83) for two plugs 87 and a tapped hole 89 in the top of the head for a conical screw 85. The two plugs 87 are optionally splined and are generally flush with the neck of the screw in the “non activated” position as illustrated in section in FIG. 8A. In this position the bone screw can be inserted through the implant hole and tightened in the bone. Once this operation is complete, the conical screw 85 is tightened, thereby causing tapered sides 88 of screw 85 to drive the plugs 87 radially outward as illustrated in section in FIG. 8B, locking the bone screw in the implant. Of course, alternative embodiments of this design can be made with one hole and plug or multiple holes and plugs as will be apparent to those having skill in the art. The holes can be located completely in the head or completely in the neck of the screw or, as illustrated, partially in the head and partially in the neck. The bone screw and conical screw can be made from metal, bioabsorbables and/or polymer and the plugs are made from a softer metal, bioabsorbables and/or polymer or an organic material such as collagen. The bone screw is inserted through the screw hole in the implant and screwed into the bone using the cruciform slots in the bone screw head. The conical screw is then screwed into the bone screw head and tightened. As the conical screw advances and the plugs are driven radially outward, they are pressed into the sides of the screw hole in the plate or one or both can slide under the plate. See, for example, FIG. 8C wherein two surgical screw assemblies 81 are used to affix bone plate 76 to a bone comprised of a cortical shell 8 and a cancellous portion 9. Two conventional screws 71 are also used to affix the bone plate to the bone. When collagen is used for the plugs, it will absorb body fluids and swell, further locking the surgical screw assemblies in place.

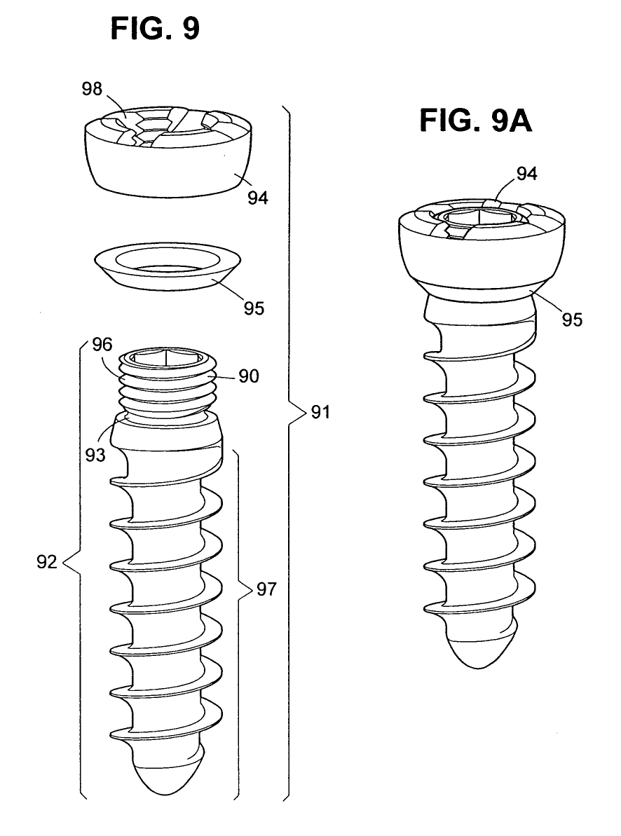

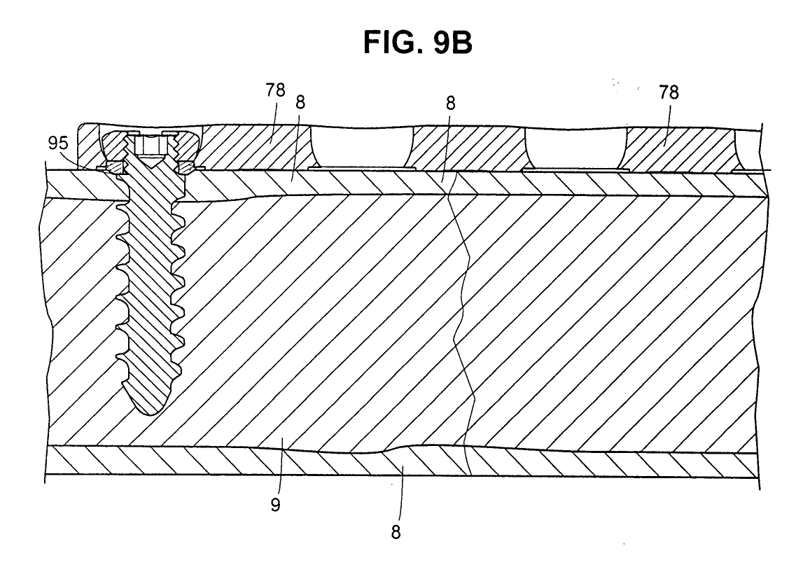

FIG. 9 is an expanded view of a crushable collar self-locking surgical screw assembly 91 comprised of a bone screw 92 which has a threaded neck 96 at the top and a collar 95 which slides over the threaded neck and seats at a bottom portion 93 of the neck. Threads 97 are arranged on the shank and a nut 94 threads onto the bone screw using the cruciform slots 98. The nut 94 when it is tightened down presses against the collar 95. The assembled screw assembly is illustrated in FIG. 9A. The bone screw and the nut can be made from metal, bioabsorbables and/or polymer while the crushable collar is made from a softer metal, bioabsorbables and/or polymer or an organic material such as collagen. The screw is inserted through a screw hole in the plate and screwed into the bone. The collar is slipped over the top of the bone screw and slid in place or can be made up as a preassembled unit with the nut not yet tightened. The nut is threaded on the top of the bone screw and tightened against the top of the collar. As the nut is tightened, the collar expands and engages the screw hole in the plate. FIG. 9B illustrates in section a plate 78 affixed to a bone, comprising a cortical shell 8 and a cancellous portion 9, with screw assembly 91. The collar 95 is pressed and somewhat deformed against the inside of the screw hole in the bone plate.

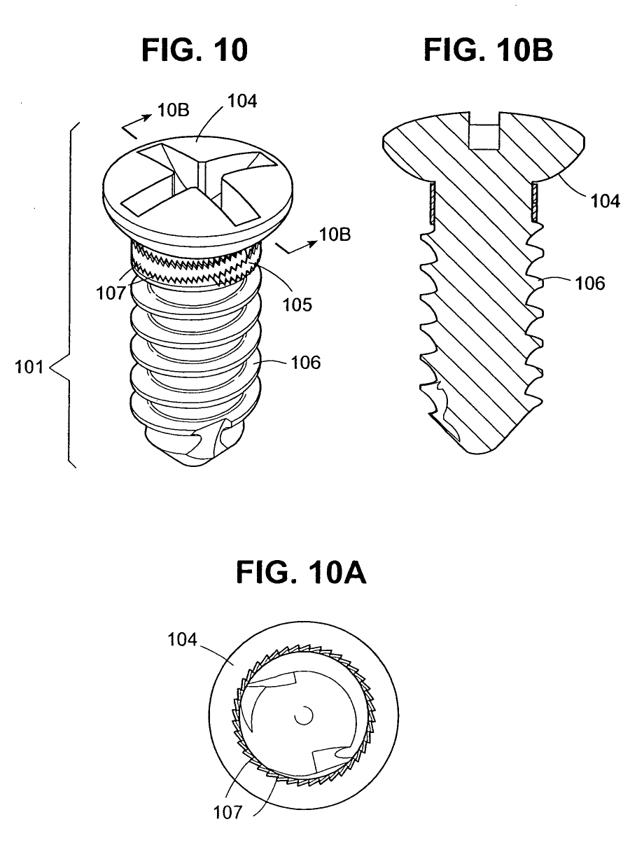

FIG. 10 is a perspective view of a locking collar type self-locking surgical screw assembly 101 having a head 104, threads 106 and a neck 105. The neck has multiple ridges 107 which are forced against the sides of the screw hole in a medical implant when the implant is installed, thereby preventing the screw from backing out. As shown, these ridges can be made in a “barb-like” fashion such that they easily deform when the screw is being tightened yet grip in the reverse direction to prevent loosening. The assembly is illustrated in section in FIG. 10B and FIG. 10A is a view from the bottom tip of the screw. The screw can be made from metal, bioabsorbables and/or polymer and the ridges can be made from metal, bioabsorbables and/or polymer or an organic material such as collagen.

What is claimed is:

1. A self-locking surgical screw assembly for fastening a medical implant to a bone comprising
a screw having a non-threaded head affixed to a shank,
a threaded portion on the shank,
a neck portion on the shank disposed between the head and the threaded portion, and
a locking device externally affixed to or disposed on the head and/or neck,
wherein the locking device is lockingly engaged with the head and/or neck of the screw when the screw is in fastening engagement with the medical implant and the bone.

2. The self-locking surgical screw assembly of claim 1 wherein the locking device is selected from the group consisting of a retaining ring, a compression collar, a plug, two or more than two plugs, a split ring, two or more than two split rings, a washer, a split washer, a tab, two or more than two tabs, a crushable collar and a multiple ridged neck.

3. The self-locking surgical screw assembly of claim 1 wherein the locking device is comprised of a bioabsorbable or resorbable material and the bioabsorbable or resorbable material optionally further comprises one or more than one bioactive material.

4. The self-locking surgical screw assembly of claim 1 wherein the locking device is comprised of an organic material such as collagen and the organic material optionally further comprises one or more than one bioactive material.

5. The self-locking surgical screw assembly of claim 1 wherein the screw is comprised of a bioabsorbable or resorbable material and the bioabsorbable or resorbable material optionally further comprises one or more than one bioactive material.

6. The self-locking surgical screw assembly of claim 5 wherein the locking device is comprised of an organic material such as collagen and the organic material optionally further comprises one or more than one bioactive material.

7. The self-locking surgical screw assembly of claim 1 wherein the screw and/or the locking device is coated with a bioabsorbable or resorbable material and the bioabsorbable or resorbable material optionally further comprises one or more than one bioactive material.

8. The self-locking surgical screw assembly of claim 7 wherein the locking device is comprised of an organic material such as collagen and the organic material optionally further comprises one or more than one bioactive material.

9. A medical implant device comprising
a self-locking surgical screw assembly for fastening a medical implant to a bone, the self-locking surgical screw assembly comprising
a screw having a non-threaded head affixed to a shank,
a threaded portion on the shank,
a neck portion on the shank disposed between the head and the threaded portion, and
a locking device externally affixed to or disposed on the head and/or neck,
and a medical implant comprising at least one untapped through hole to accommodate the self-locking screw assembly,
wherein the locking device is lockingly engaged with the head and/or neck of the screw when the screw is in fastening engagement with the medical implant and the bone.

10. The device of claim 9 wherein the locking device is selected from the group consisting of a retaining ring, a compression collar, a plug, two or more than two plugs, a split ring, two or more than two split rings, a washer, a split washer, a tab, two or more than two tabs, a crushable collar and a multiple ridged neck.

11. The device of claim 9 wherein the medical implant is selected from the group consisting of a bone plate, an artificial joint and an acetabulum cup.

12. A medical implant device affixed to a bone of a surgical patient comprising
a medical implant comprising at least one untapped through hole,
a bone of a surgical patient, and
a self-locking screw assembly affixed in the at least one untapped through hole and fastening the medical implant to the bone, the self-locking surgical screw assembly comprising
a screw having a non-threaded head affixed to a shank,
a threaded portion on the shank,
a neck portion on the shank disposed between the head and the threaded portion, and
a locking device externally affixed to or disposed on the head and/or neck,
wherein the locking device is lockingly engaged with the head and/or neck of the screw when the screw is in fastening engagement with the medical implant and the bone.

13. The device of claim 12 wherein the locking device is selected from the group consisting of a retaining ring, a compression collar, a plug, two or more than two plugs, a split ring, two or more than two split rings, a washer, a split washer, a tab, two or more than two tabs, a crushable collar and a multiple ridged neck.

14. The device of claim 12 wherein the medical implant is selected from the group consisting of a bone plate, an artificial joint and an acetabulum cup.

15. A method of affixing a medical implant to a bone comprising fastening the medical implant to the bone with a self-locking surgical screw assembly of claim 1.
